(19) United States
(12) Patent Application Publication
(10) Pub. No.: US 2022/0280027 A1
(43) Pub. Date: Sep. 8, 2022

(54) IMAGE CAPTURING MODULE, ENDOSCOPE WITH IMAGE CAPTURING MODULE, AND METHOD OF MANUFACTURING SAME

(71) Applicant: OLYMPUS CORPORATION, Tokyo (JP)

(72) Inventor: Shigeru HOSOKAI, Tokyo (JP)

(73) Assignee: OLYMPUS CORPORATION, Tokyo (JP)

(21) Appl. No.: 17/573,750

(22) Filed: Jan. 12, 2022

Related U.S. Application Data
(60) Provisional application No. 63/155,335, filed on Mar. 2, 2021.

(51) Int. Cl. A61B 1/00; H04N 5/225; A61B 1/05

(52) U.S. Cl. CPC A61B 1/00096; H04N 5/2254; H04N 5/2253; A61B 1/05; A61B 1/0011; H04N 2005/2255

(57) ABSTRACT

Image capturing module with imager module including lens and image sensing circuitry is fixed within a recess of a frame by resin between the imager module and surface(s) of the recess. The top surface of the lens has a surface feature(s), structural feature(s) or surface modification(s) that, during a process of disposing the resin, prevents the resin from covering a central region of the top surface of the lens. Surface features (e.g., a groove), structural features (e.g., a groove or a protrusion or a strip of material), and surface modifications (e.g., change in wettability or difference in surface chemistry with respect to the resin) are suitably located on top surface of lens to maintain an area not covered by resin that allows sufficient light propagation through lens for operability of Image capturing module. Methods of manufacture of such image capturing modules are also disclosed.

### RELATED APPLICATION DATA

This application is based on and claims priority under 35 U.S.C. § 119 to U.S. Provisional Application No. 63/155,335 filed on Mar. 2, 2021, the entire contents of which are incorporated herein by reference.

### FIELD OF DISCLOSURE

The present disclosure relates to an imaging unit, and more particularly, to an imaging unit for an endoscope.

### BACKGROUND

An endoscope may serve as a device for observation, such as in a medical procedure, where the endoscope may be inserted into a body (human or otherwise) and images obtained therefrom.

Endoscopes may employ an image sensor (or “imager”) to capture an image inside the body and communicate the image to an external system via, e.g., electronic signals. Such an imager may be included as part of an imager module at the distal end of the endoscope. Among other elements, the imager module may further include a laminated lens on the imager to focus the image thereon.

Some endoscopes, or the imager modules at the distal ends thereof, are made to be disposable. For this and other reasons, it may be preferable that they be manufactured at a low cost.

However, during manufacturing, mechanical damage may occur on end surfaces of a laminated lens included in the imager. For example, in the process of manufacturing the laminated lens, a plurality of glass substrates each having a lens may be bonded to form a wafer of laminated layers. A mechanical dicer may then be used to cut the wafer into individual laminated lenses for respective imagers. This dicing may cause chipping and fragmentation to occur on the end surfaces of the laminated lens. Such damage may cause a decrease in optical quality, as well as pose a risk of fragments falling off after manufacturing. Such fragments falling off during use may be particularly detrimental, particularly when used in medical procedures.

Japanese Laid-open Publication No. 2008-182051 describes an optical device including a sealing resin and transparent member. According to the publication, it is preferable to fill the sealing resin slightly below the upper surface of the transparent member. Thus, the upper portion of the side surfaces of the transparent member is exposed.

Japanese Laid-open Publication No. 2004-304081 describes a semiconductor chip with an alleged increased adhesive strength between a chip end face and a resin, in order to suppress chipping of a chip angle or peeling of the resin. The end surface of the semiconductor chip is formed of a series of curved surfaces. According to the publication, the series of curved surfaces provides a larger contact area between the chip end face and the resin to increase adhesion.

### SUMMARY

Accordingly, there is a need for an imaging unit that can substantially obviate one or more of the issues due to limitations and disadvantages of the related art.

An object of the present disclosure is to provide an imaging unit having a lens whose end surfaces, which may be damaged, are prevented from causing deleterious effects such as fragmentation or a decrease in optical quality.

Additional features and advantages will be set forth in the description that follows, and in part will be apparent from the description, or may be learned by practice of the disclosure. The objectives and other advantages disclosed herein will be realized and attained by the structure particularly pointed out in the written description and claims thereof, as well as the appended drawings.

Exemplary embodiments of an image capturing module comprise a frame including a bottom surface and sidewalls, the bottom surface and sidewalls defining a recess, an imager module including a lens and image sensing circuitry, wherein the imager module is disposed within the recess of the frame, and a resin between the imager module and at least one of the sidewalls of the recess. A region of a top surface of the lens includes a surface feature or a surface modification that, during a process of disposing the resin, prevents the resin from covering a central region (C) of the top surface of the lens and a wiring pattern of the image sensor of the endoscope is connected to the observation device via a circuit that includes the wiring pattern.

Exemplary embodiments of methods of manufacturing an image capturing module including an imager module with a lens and image sensing circuitry comprises modifying a portion of a top surface of the lens to include a surface feature or a surface modification, placing the imager module in a recess of a frame, the recess having a bottom surface and sidewalls, and filling a gap between side surfaces of the lens and sidewalls of the frame with a resin. The resin contacts the surface feature or the surface modification, and the surface feature or the surface modification prevents the resin from flowing onto a central region of the top surface of the lens

Example surface features include a groove at each edge where the top surface of the lens meets each side surface of the lens and a structural feature, such as a groove or a protrusion on the top surface of the lens or a strip of material disposed on the top surface of the lens, that is, in some embodiments, offset from at least one edge of the top surface. Example surface modifications include an area of increased wettability to the resin that is located at a periphery of the top surface of the lens or an area having a different surface chemistry, particularly with respect to the resin, as compared to a central region of the top surface.

In additional aspects, the image capturing module is seated in a recess of a cartridge body and is configured to be attached to a distal end of a medical device, such as an endoscope. The medical device can be incorporated into an imaging system that includes the medical device, e.g., the endoscope having the disclosed image capturing module, an observation device, and a display device.

It is to be understood that both the foregoing general description and the following detailed description are exemplary and explanatory and are intended to provide further explanation of the disclosure as claimed.

### BRIEF DESCRIPTION OF THE DRAWING

The accompanying drawings, which are included to provide a further understanding of the disclosure and are incorporated in and constitute a part of this application, illustrate embodiments of the disclosure and together with the description serve to explain principles of the disclosure. In the drawings:

FIG. 1 illustrates a configuration of a medical observation system according to an example embodiment.

FIG. 2 illustrates an imaging unit according to an example embodiment attached to an endoscope.

FIG. 3 illustrates an imaging unit according to an example embodiment.

FIG. 4 illustrates a perspective view of an image capturing module according to an example embodiment.

FIG. 5 illustrates a cross-section of the image capturing module taken at line A-A′ of FIG. 4.

FIG. 6 show a method of manufacturing an image capturing module according to an example embodiment of the present disclosure.

FIG. 7 illustrates an imager module according to an example embodiment of the present disclosure.

FIGS. 8A and 8B show aspects of a method of manufacturing an image capturing module according to an example embodiment of the present disclosure.

FIGS. 9A and 9B show aspects of a method of manufacturing an image capturing module according to another example embodiment of the present disclosure.

FIGS. 10A and 10B show aspects of a method of manufacturing an image capturing module according to a further example embodiment of the present disclosure.

Throughout all of the drawings, dimensions of respective constituent elements are appropriately adjusted for clarity. For ease of viewing, in some instances only some of the named features in the figures are labeled with reference numerals.

### DETAILED DESCRIPTION

Reference will now be made in detail to embodiments of the present disclosure, examples of which are illustrated in the accompanying drawings.

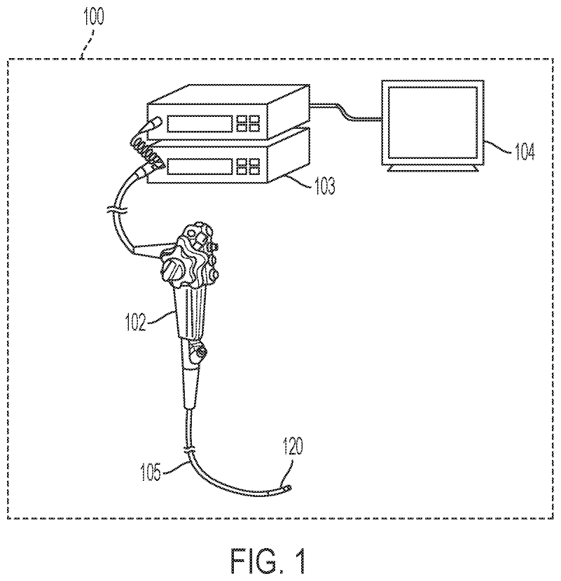

FIG. 1 is a block diagram illustrating a configuration of a medical observation system 100 according to an example embodiment. As illustrated in FIG. 1, a medical observation system 100 may include an endoscope 102 that transmits images obtained by imaging unit 120 from within a living subject. In an example, imaging unit 120 transmits these images via electrical signals via circuitry contained within the endoscope 102 to an observation device 103. The observation device 103 may recreate the image based on the electrical signals, and a display device 104 connected to the observation device 103 may display the recreated image generated by the observation device 103. The display device 104 may include a display panel such as a liquid crystal display, an organic electroluminescence (EL) display, or the like.

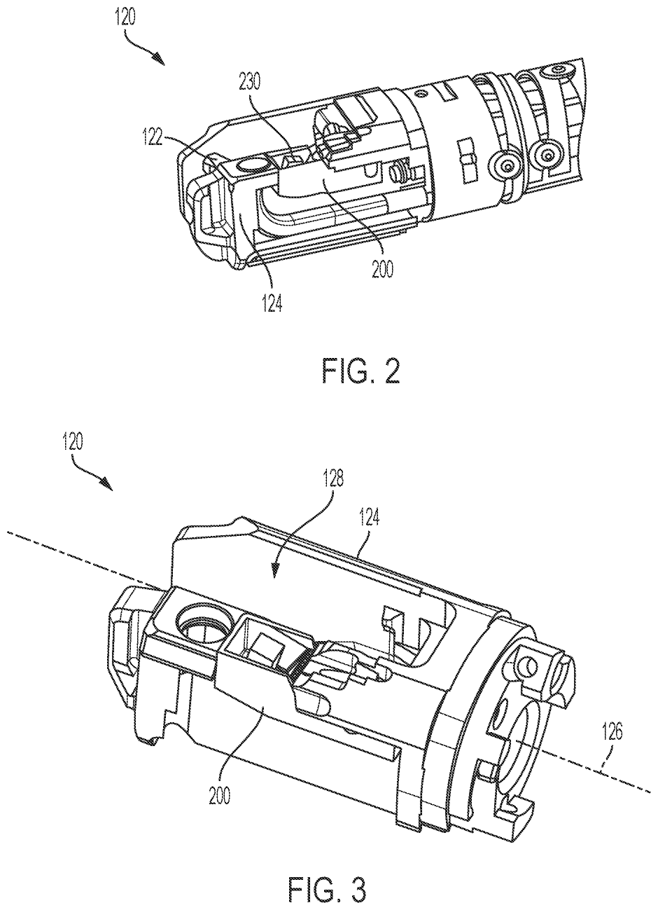

FIGS. 2 and 3 each illustrate perspective, cut-away views of an imaging unit 120 according to an example embodiment of the present disclosure. The imaging unit 120 may be at the distal end of endoscope 102, for example attached to the distal end of a flexible insertion tube 105 of an endoscope as shown in FIG. 1, and may be inserted into a patient to obtain images (e.g., pictures or video) therein. The term “patient,” as used herein, comprises any and all organisms and includes the term “subject.” A patient can be a human or an animal.

In some embodiments, the imaging unit 120 may be detachable from the endoscope 102 such that it may be easily disposed of and replaced. In other embodiments, the entire endoscope 102 may be disposable.

As shown by example in FIGS. 2 and 3, the imaging unit 120 may include a cartridge body 124 that may provide structural support for an image capturing module 200 housing the imaging unit 120. In some embodiments, the cartridge body 124 may be made of a metal such as steel (e.g., medical-grade steel). The image capturing module 200 is seated in a recess of the cartridge body 124. The recess may be offset from a centerline or center axis 126 of the cartridge body 124 so as to allow an open space 128 in the cartridge body 124 to accommodate other structures and functionality of the endoscope, such as additional lumens for insertion and removal or surgical tools, optical equipment, and/or irrigation. In some aspects, the cartridge body 124 supports a movable portion 122, such as disclosed in U.S. Pat. No. 10,321,806, the entire contents of which are incorporated herein by reference.

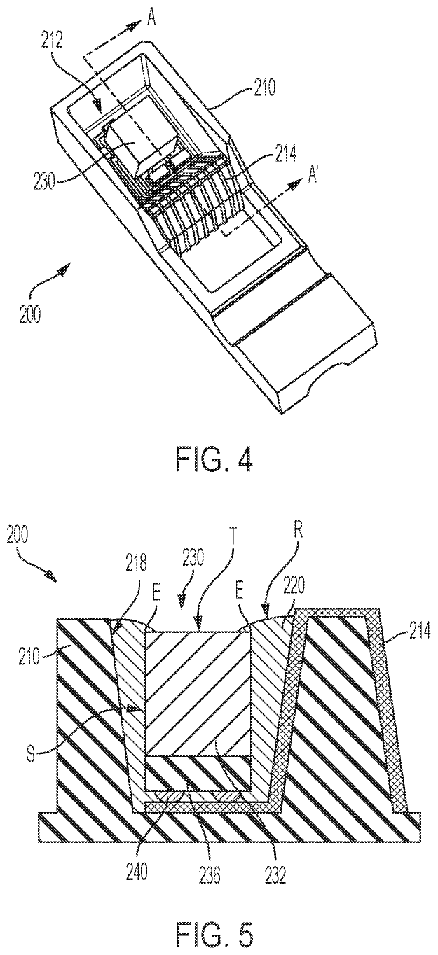

FIG. 4 illustrates a perspective view of the image capturing module 200 according to an example embodiment, and FIG. 5 shows a cross-section of the capturing module 200 taken at line A-A′ of FIG. 4 (with the addition of a resin). As shown in FIGS. 4 and 5, the image capturing module 200 may include a frame 210. In example embodiments, the frame 210 is formed by, e.g., injection-molded resin or other material (i.e., a “molded frame”). The frame 210 includes a bottom surface and sidewalls 218, which define a recess 212. An imager module 230 is seated in the recess 212. The imager module 230 includes a lens 232 and image sensing circuitry (“imager”) 236. The lens 232 includes one or more glass substrates each having side surfaces extending substantially perpendicularly from the top surface (T). In some embodiments, the lens 232 may be a laminated lens formed of multiple layers of glass substrates that are, for example, bonded together. The image sensing circuitry 236 may be disposed underneath the lens 232 and may be bonded to the lens 232, for example, by gluing tougher with clear resin. In an example embodiment, the image sensing circuitry 236 may include an image sensor for converting light to electrical signals, and may transmit these electrical signals to observation device 103 via a wiring pattern 214, which may include one or more wires composed of a conductive metal such as, for example, copper, gold, or silver. In some embodiments, the image sensing circuitry 236 may connect to the wiring pattern 214 via solder joints 240. The solder joints 240 may take the form of a ball grid array on the image sensing circuitry 236, but embodiments are not limited thereto. In some aspects, the image sensing circuitry 236 is embodied in a semiconductor substrate with electrical circuits and electrical components formed therein by, for example, photolithographic techniques, and the lens is bonded to a surface of the substrate.

As will be discussed in more detail below, FIG. 5 illustrates a resin 220 (e.g., a sealing adhesive) that may be filled around the imager module 230 in the recess 212 of the frame 210, such that the resin 220 is disposed between the imager module 230 and at least one sidewall 218 of the frame 210, alternatively between the imager module 230 and all sidewalls 218 of the frame 210 or between the imager module 230 and the bottom and the sidewalls 218 of the frame 210, to fix the imager module 230 in the recess 212. In some aspects, the resin 220 is optically non-transparent, for example is black when cured. In other aspects, the resin 220 is optically transparent. In exemplary embodiments, where the resin is optically transparent, the outer circumference of the top surface (T) and the side surfaces (S) of the lens 232 and the side surfaces of the image sensing circuitry 236 are covered with a light-shielding element. Therefore, it is generally desirable to use black that does not transmit light. In particular aspects, the resin 220 is located in the gap between the side surfaces (S) of the imager module 230 and the sidewall(s) 218.

The top surface (T) of the lens 232 meets each side surface (S) at an edge (E) and, in some aspects, the resin 220 fills the gap to a height such that a top surface (R) of the resin 220 meets the edge (E). In other aspects, the resin 220 fills the gap to a height such that a top surface (R) of the resin 220 is above the edge (E) and extends onto the peripheral region (P) of the top surface (T) so as to leave a central region (C) of the top surface (T) of the lens 232 uncovered by resin 220. In other words, the central region (C) is inward of the peripheral region (P), which may be coated or encapsulated by the resin 220. In some exemplary embodiments, the size of the central region (C) is considered as having a specified area. For example, the central region (C) of the top surface (T) is at least 80% of a total area of the top surface (T) of the lens 232. In alternative embodiments, the central region (C) of the top surface (T) is at least 90% or at least 95% of a total area of the top surface (T) of the lens 232. In other exemplary embodiments, the size of the central region (C) is considered as that appropriate to allow sufficient incident light to propagate into the lens 232 so that the imager module 230 functions. As describe herein, a region of a top surface (T) of the lens 232 (such as a peripheral region (P) or an interface between the peripheral region (P) and the central region (C)) can include a surface feature or a surface modification that prevents the resin 220 from covering the central region (C) of the top surface (T) of the lens 232. Such surface features and surface modifications can be physical and/or chemical features, or a combination thereof.

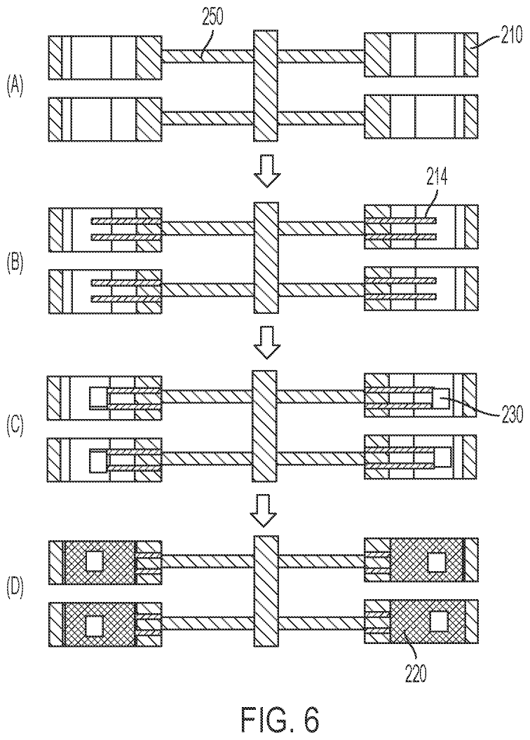

FIG. 6 illustrates a method of manufacturing image capturing modules 200 according to an example embodiment of the present disclosure. With reference to part (A) of FIG. 6, multiple frames 210 (for illustrative purposes, four) are provided that may have been formed by, for example, injection-molded resin. In FIG. 6, such frames 210 are shown still attached to the runners 250 from the injection molding process. After the frames 210 have been formed, and as shown in part (B), a conductive material such as metal (e.g., gold, copper, or silver, but not limited thereto) is patterned (e.g., by masking and etching) to create wiring patterns 214. Then, as shown in part (C), imager modules 230 are mounted on the wiring patterns 214 in recesses 212 of the frames 210. For example, as discussed above, the imager modules 230 may be soldered to the wiring patterns 214 using, e.g., a ball grid array 240. Finally, in part (D), the gap between the imager modules 230 and the frames 210 is filled with resin 220 to fix the imager modules 230 within the recesses of the frames 210. The resin 220 is then hardened by, e.g., curing, which can utilize heat, radiation, electron beams, or chemical additives.

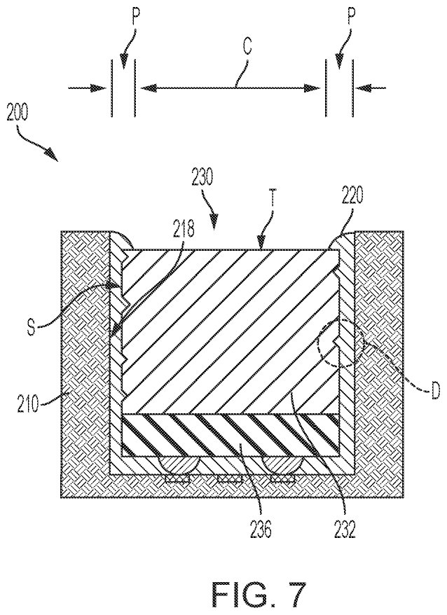

When the gap between an imager module 230 and frame 210 is filled with resin 220, as discussed above, and as illustrated by example in FIG. 7, edges (E) and peripheral regions (P) of the top surface (T) of lens 232 may also be coated with the resin 220 such that they are encapsulated by the resin 220 and are not exposed (while leaving central region (C) uncoated by resin 220). Thus, even if the end surfaces of the individual layers of lens 232 (e.g., end surfaces exposed at the side surface (S) of the lens 232) and/or edges (E) of the lens 232 include mechanical damage, such as chipping and/or fragmentation (an example of which is schematically illustrated at region (D) in FIG. 7), the resin 220 may prevent such damage from having an impact on optical quality of the lens 232 or detachment of the fragments.

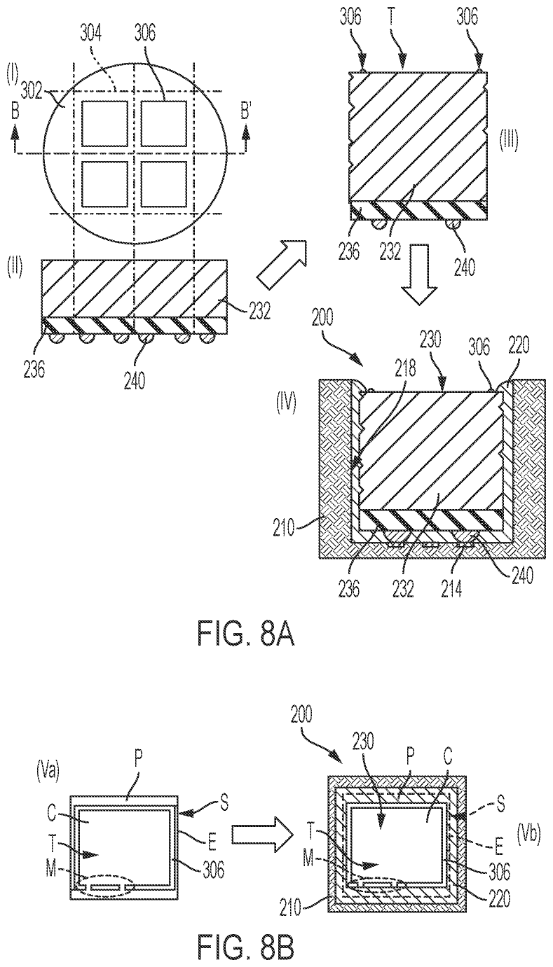

FIG. 8A illustrates a detailed example of forming an image capturing module 200 according to an example embodiment of the present disclosure. As shown in FIG. 8A, the imager module 230 is placed in a frame 210 to be surrounded by resin 220, an example process for which was described above with reference to FIG. 6. Prior to this placement, however, and as illustrated in FIG. 8A part (I), scribe lines 304 are made on a top surface of a lens wafer 302 to indicate where to cut or dice the lens wafer 302 into individual imager modules 230. This lens wafer 302 may include one or more layers of glass bonded together, as well as image sensing circuitry 236. The image sensing circuitry 236 is disposed underneath and affixed to the respective lenses 232, preferably prior to cutting/dicing into individual imager modules 230. However, the lens wafer 232 and the circuit wafer can also be individually cut or diced and then individually bonded. Illustrated in part (II), which is a cross section taken at line B-B′ of part (I), is the assembled lenses 232 and image sensing circuitry 236 as well as solder joints 240 in the uncut imager module assembly and showing the scribe lines 304 through the thickness of the assembly.

Furthermore, a structural feature, such as a protrusion 306, may be present which protrudes from or is formed on the top surface of lens wafer 302 to border an inside perimeter of the end surfaces of the lens wafer 302. In the cross-sectional side view of part (III) of FIG. 8A, only the protrusions 306 on two of the four sides of the exemplary quadrilateral imager module 230 are shown. However and as shown in a top view in part (Va) and part (Vb) in FIG. 8B, the protrusion 306 may form a circumference of a closed area, i.e., the central region (C) on the top surface (T), and functions as a barrier or dam to prevent the resin from covering all or part of the central region (C) of the top surface (T) of the lens 232. Part (Va) in FIG. 8B is a top view corresponding to part (III) in FIG. 8A, and part (Vb) in FIG. 8B is a top view corresponding to part (IV) in FIG. 8A. The arrow in FIG. 8B from part (Va) to part (Vb) is indicative of processing by which the imager module 230 is disposed within the recess 212 of the frame 210 and resin 220 is added to the gap between the imager module, e.g., the side surface (S), and the at least one or more sidewalls 218 of the frame 210 such that the resin 220 coats edges (E) and peripheral regions (P) of the top surface (T) of lens 232.

In some aspects, the circumference formed by the protrusion 306 is a continuous structure, but discontinuous structures may also be used as long as any discontinuities still allow the protrusion 306 forming the circumference to function as a barrier or dam to prevent the resin from covering all or part of the central region (C) of the top surface (T) of the lens 232 and that the imaging function of the imager module is retained. Example discontinuous structures for the protrusion 306 are shown at region (M) in part (Va) and part (Vb) of FIG. 8B. The protrusion 306 may be formed by a printing technology. In some embodiments, the protrusion 306 can be a suitable material that is sufficiently workable to be applied to the lens 232, sufficiently adheres to the lens 232 and functions as a dam to the resin intruding onto the central region (C), and is biocompatible.

As noted above, after the wafer 302 is diced, each imager module 230 may be placed within a frame 210 and soldered or otherwise placed in electrical contact with wiring pattern 214. Then, as shown in cross-sectional view in part (IV) of FIG. 8A, the resin 220 may fill the gap between the imager module 220 and interior surfaces of the recess of the frame 210 as was described with reference to FIG. 6. Furthermore, in this example embodiment, the resin 220 is filled until it overflows from the gap and begins to flow onto the peripheral regions (P) of the top surface (T), thereby covering chips and/or fragmentations and/or other defects on the side surface (S) and edge (E) of the lens 232. Meanwhile, the protrusion 306 acts as a barrier and prevents the resin 220 from flowing further than the peripheral regions (P) of the top surface (T). Therefore, a central region (C) of the top surface (T) of the lens 232 of imager module 230, whose outer perimeter may be defined by the protrusion 306, may be free of resin 220. The resin 220 is then cured to secure the imager module 230 in the frame 210, while also preventing a reduction in optical quality or fragmentation at the edges of the image module 230.

In some embodiments, the protrusion 306 remains in the final product. However, embodiments are not limited thereto, and in other embodiments, the protrusion 306 may be removed, e.g., by selective etching, after the resin 220 is sufficiently cured.

Although shown and described in FIGS. 8A and 8B as a protrusion 306, the structural feature can also take other forms. For example, the structural feature can be a groove that protrudes into or is formed in the top surface of lens wafer 302 to border an inside perimeter of the end surfaces of the lens wafer 302. Also for example, the structural feature can be a strip of material that is affixed (permanently or temporarily) to the top surface of lens wafer 302 to border an inside perimeter of the end surfaces of the lens wafer 302. Such grooves and strips can be used to the same effect and functionality as disclosed with respect to the protrusion 306.

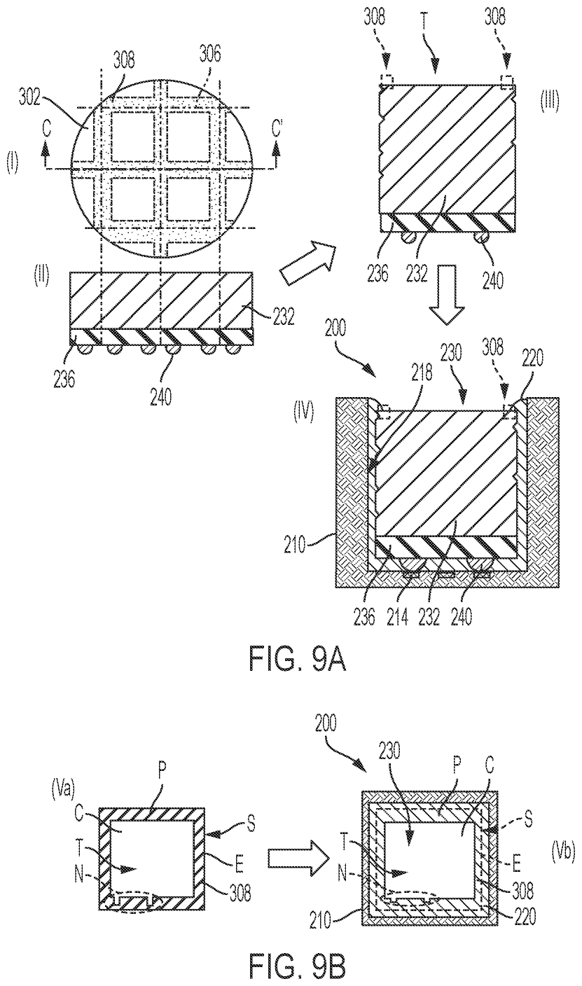

FIGS. 9A and 9B illustrate a detailed example of forming an image capturing module 200 according to another example embodiment of the present disclosure. As shown in FIGS. 9A and 9B, the imager module 230 is placed in a frame 210 to be surrounded by resin 220, an example process for which was described above with reference to FIG. 6. Prior to this placement, however, and as illustrated in FIG. 9A part (I), scribe lines 304 are made on a top surface of a lens wafer 302 to indicate where to cut or dice the lens wafer 302 into individual imager modules 230. This lens wafer 302 may include one or more layers of glass bonded together, as well as image sensing circuitry 236. The image sensing circuitry 236 is disposed underneath and affixed to the respective lenses 232, preferably prior to cutting/dicing into individual imager modules 230. Illustrated in part (II), which is a cross section taken at line C-C′ of part (I), is the assembled lenses 232 and image sensing circuitry 236 as well as solder joints 240 in the uncut imager module assembly and showing the scribe lines 304 through the thickness of the assembly.

In this example embodiment, at least the top surface in regions 308 of the lens wafer 302 overlapping the scribe lines 304 includes a surface modification. In some aspects, the surface modification changes a surface chemistry of a peripheral region (P) to differ from that of the central region (C). For example, the surface modification can increase wettability to the resin 220 of the treated area of the top surface as compared to wettability to the resin 220 of untreated areas of the top surface. In another example, the surface modification can increase an affinity between the resin 220 and the treated area of the top surface to the resin 220 as compared to an affinity between the resin 220 and the central region (C). In yet another example, the surface modification can change the chemical potential of the treated area of the top surface (T), such as the peripheral region (P), as compared to the chemical potential of the untreated area of the top surface (T), such as the central region (C), so that the resin 220 preferentially flows and coats the treated area but does not flow to or coat the untreated area.

As an example, at least the top surface (T) in regions 308, alternatively, all of the top surface (T) in regions 308, may be chemically coated or otherwise modified to improve wettability. As another example, at least the top surface (T) in regions 308, alternatively, all of the top surface (T) in regions 308, may be roughened to enhance adhesion of the resin. Examples of techniques and processes to improve wettability include surface modification methods, such as UV irradiation, plasma processing, frame processing, etc, as well as those available from, for example, K. BRASCH & CO., LTD. and ASUMI GIKEN, Limited.

In the cross-sectional side view of part (III) of FIG. 9A, only the regions 308 on two of the four sides of the exemplary quadrilateral imager module 230 are shown. However and as shown in top view in part (Va) and part (Vb) in FIG. 9B, the treated surface in the regions 308 can form a circumference of a closed area, i.e., the central region (C) on the top surface (T). Because the resin will preferentially remain in the treated area in regions 308, the treated area functions as a barrier to prevent the resin from covering all or part of the central region (C) of the top surface (T) of the lens 232. Part (Va) in FIG. 9B is a top view corresponding to part (III) in FIG. 9A and part (Vb) in FIG. 9B is a top view corresponding to part (IV) in FIG. 9A. The arrow in FIG. 9B from part (Va) to part (Vb) is indicative of processing by which the imager module 230 is disposed within the recess 212 of the frame 210 and resin 220 is added to the gap between the imager module 230, e.g., the side surface (S), and the at least one or more sidewalls 218 of the frame 210 such that the resin 220 coats edges (E) and peripheral regions (P) of the top surface (T) of lens 232, but only for treated areas in regions 308.

In some aspects, the circumference formed by the treated areas in regions 308 is a continuous structure, but discontinuous structures may also be used as long as any discontinuities still allow the treated areas in regions 308 forming the circumference to function as a barrier to prevent the resin 220 from covering all or part of the central region (C) of the top surface (T) of the lens 232 and that the imaging function of the imager module is retained. Example discontinuous structures for the treated areas in regions 308 are shown at region (N) in part (Va) and part (Vb) of FIG. 9B.

As noted above and similar to the example embodiment described with reference to FIGS. 8A and 8B, after the wafer 302 is diced, each imager module 230 may be placed within a frame 210 and soldered to wiring pattern 214. As shown in part (IV) of FIG. 9A, the resin 220 may fill the gap between the imager module 220 and interior surfaces of the recess of the frame 210 as was described with reference to FIG. 6. Furthermore, in this example embodiment, the resin 220 is filled until it overflows from the gap and begins to flow onto treated area of regions 308 at the peripheral regions (P) of the top surface (T), thereby covering chips and/or fragmentations and/or other defects on the side surface (S) and edge (E) of the lens 232. For example, the resin 220 may flow onto regions of the surface of the lens 232 that have been treated to have a chemical or structural affinity for the resin such that the resin will flow and cover those treated regions while note flowing to or covering non-treated regions. In some aspects, surface tension of the resin, in combination with the surface modification, may contribute to preventing the resin 220 from flowing onto untreated regions, e.g., a central region (C) of the top surface (T) of the lens 232 that has not been treated. The resin 220 is then cured to secure the imager module 230 in the frame 210, while also preventing a reduction in optical quality or fragmentation at the edges of the image module 230.

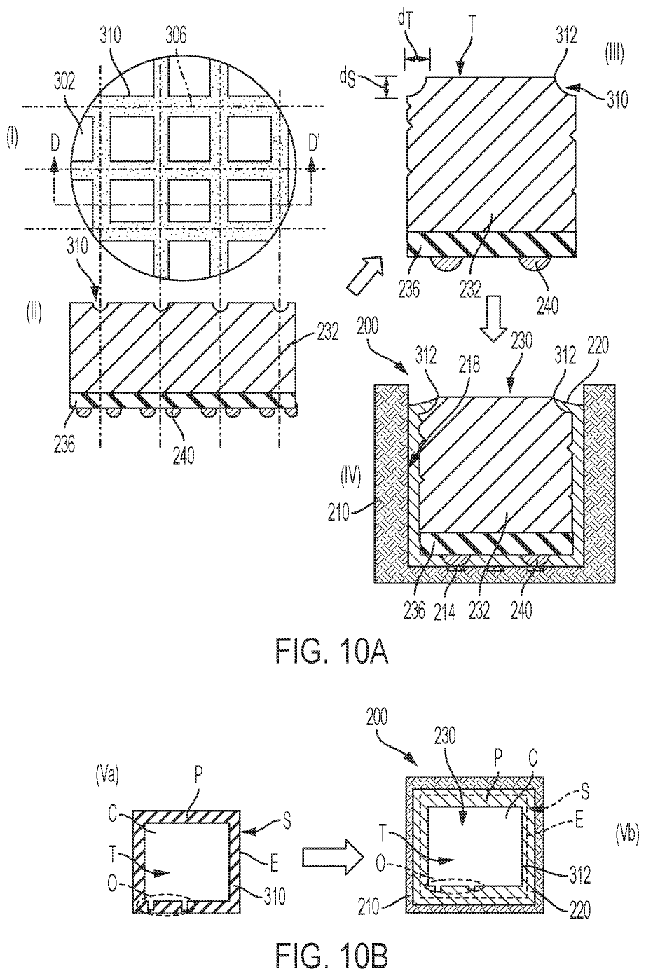

FIGS. 10A and 10B illustrate a detailed example of forming an image capturing module 200 according to a further example embodiment of the present disclosure. As shown in FIGS. 10A and 10B, the imager module 230 is placed in a frame 210 to be surrounded by resin 220, an example process for which was described above with reference to FIG. 6. Prior to this placement, however, and as illustrated in FIG. 10A parts (I) and (II), grooves 310 are formed in the wafer 302 by, e.g., patterning and etching. Before or after these grooves 310 are formed, scribe lines 304 may be formed on the surface of wafer 302 to indicate where to cut or dice the lens wafer 302 into individual imager modules 230. The lens wafer 302 may include one or more layers of glass bonded together, as well as image sensing circuitry 236. The image sensing circuitry 236 is disposed underneath and affixed to the respective lenses 232, preferably prior to cutting/dicing into individual imager modules 230. Illustrated in part (II), which is a cross section taken at line D-D′ of part (I), is the assembled lenses 232 and image sensing circuitry 236 as well as solder joints 240 in the uncut lens wafer 302 assembly and showing the scribe lines 304 through the thickness of the assembly.

After the wafer 302 is cut or diced into individual imager modules 230, the grooves 310 are resultantly located at end surfaces of the top of the lens 232 of each imager module 230. It should be noted that while the method described above forms the grooves before cutting or dicing, embodiments are not limited thereto, and in other embodiments, the grooves 310 may be formed after the wafer is cut or diced.

In this example embodiment, the structural feature in the form of a groove 310 protrudes into or is formed in the top surface of lens wafer 302 and forms a border at a perimeter of the top surface (T) and side surfaces (S) of the lens wafer 302. In the individual imager modules 230, the groove 310 is formed into the body of the lens wafer 302 and has dimensions along both the side surfaces (S) and the top surface (T), labeled as d_{S} and d_{T}, respectively. In the cross-sectional side view of part (III) of FIG. 10A, only the grooves 310 on two of the four sides of the exemplary quadrilateral imager module 230 are shown. However and as shown in top view in part (Va) and part (Vb) in FIG. 10B, the groove 310 forms a circumference of a closed area, i.e., the central region (C) on the top surface (T), and functions as a barrier or dam to prevent the resin from covering all or part of the central region (C) of the top surface (T) of the lens 232. Part (Va) in FIG. 10B is a top view corresponding to part (III) in FIG. 10A and part (Vb) in FIG. 10B is a top view corresponding to part (IV) in FIG. 10A. The arrow in FIG. 10B from part (Va) to part (Vb) is indicative of processing by which the imager module 230 is disposed within the recess 212 of the frame 210 and resin 220 is added to the gap between the imager module, e.g., the side surface (S), and the at least one or more sidewalls 218 of the frame 210 such that the resin 220 is present in at least a portion of the groove, alternatively the resin 220 coats the entire surface of the groove 310 and an edge of the resin is coincident with the interface where the surface of the groove 310 meets the top surface (T), i.e., location 312 shown in part (III) and part (IV) of FIG. 10A.

In some aspects, the circumference formed by the groove 310 is a continuous structure, but discontinuous structures may also be used as long as any discontinuities still allow the groove 310 forming the circumference to function as a barrier or dam to prevent the resin from covering all or part of the central region (C) of the top surface (T) of the lens 232 and that the imaging function of the imager module is retained. Example discontinuous structures for the groove 310 are shown at region (0) in part (Va) and part (Vb) of FIG. 10B.

As noted above and similar to the example embodiment described with reference to FIGS. 8A and 8B and FIGS. 9A and 9B, after the wafer 302 is cut or diced, each imager module 230 may be placed within a frame 210 and soldered to wiring pattern 214.

In further embodiments, the resin 220 is filled in the gap until the resin 220 completely covers the surfaces of grooves 310, thereby covering chips and/or fragmentations and/or other defects on the side surface (S) and edge (E) of the lens 232. However, in exemplary embodiments, the resin 220 does not flow onto the top surface (T) of the imager module 230. Then, the resin 220 is solidified to secure the imager module 220 in the frame 210, while also preventing a reduction in optical quality or fragmentation at the edges of the image module 230.

In some embodiments, depending on chemical properties of the resin 220 during filling the gap and changes during curing, the surface of the cured resin 220 may remain below a plane containing the top surface (T).

In still further embodiments, both surface features and surface modification features may be combined in the same image capturing module 200, with some or all of the features present that are associated with embodiments disclosed in relation to FIGS. 8A-B, FIGS. 9A-B and/or FIGS. 10A-B.

Although the lens 232 is illustrated having the shape of a cube with a planar top surface (T) and planar side surfaces (S), the lens 232 can have alternative shapes, such as a cylinder with a circular top surface and a single side surface, or a polygon with a polygonal top surface and N side surfaces (with N-sides corresponding to the type of polygon, with N equal to 3 or more). In some embodiments, the side surfaces (S) are planar and perpendicular relative to the top surface (T) and/or the substrate of the image sensing circuitry 236; in other embodiments, the side surfaces (S) are angled relative to the top surface (T) and/or the substrate of the image sensing circuitry 236. Also, in some embodiments, the side surfaces (S) are planar; in other embodiments, the side surfaces (S) are convex, concave or a combination thereof. The presence of mechanical damage, such as chipping and/or fragmentation (an example of which is schematically illustrated at region (D) in FIG. 7), is not considered in characterizing the shapes and geometric relationships of the lens surfaces.

Although the present disclosure has been described in connection with example embodiments thereof, it will be appreciated by those skilled in the art that additions, deletions, modifications, combinations, and substitutions not specifically described may be made without department from the spirit and scope of the disclosure as defined in the appended claims. Thus, it is intended that the present invention cover the additions, deletions, modifications, combinations, and substitutions of this disclosure provided they come within the scope of the appended claims and their equivalents.

What is claimed is:

1. An image capturing module (200), comprising:
a frame (210) including a bottom surface and sidewalls (218), the bottom surface and sidewalls defining a recess (212);
an imager module (230) including a lens (232) and image sensing circuitry (236), wherein the imager module is disposed within the recess of the frame; and
a resin (220) between the imager module and at least one of the sidewalls of the recess;
wherein a region of a top surface (T) of the lens includes a surface feature or a surface modification that, during a process of disposing the resin, prevents the resin from covering a central region (C) of the top surface of the lens.

2. The image capturing module according to claim 1, wherein the lens includes a glass substrate having side surfaces extending substantially perpendicularly from the top surface, and wherein the lens is bonded to a surface of a substrate containing the image sensing circuitry.

3. The image capturing module according to claim 1, wherein an area of the central region of the top surface of the lens is at least 80%, preferably at least 90%, more preferably at least 95%, of a total area of the top surface of the lens.

4. The image capturing module according to claim 1, wherein the lens includes one or more side surfaces, wherein the top surface meets each side surface at an edge, and wherein the surface feature includes a groove at each edge.

5. The image capturing module according to claim 4, wherein the groove surrounds the central region of the top surface.

6. The image capturing module according to claim 1, wherein the surface feature includes a structural feature on the top surface of the lens.

7. The image capturing module according to claim 6, wherein the top surface meets each side surface at an edge, and wherein the structural feature is offset from at least one edge of the top surface.

8. The image capturing module according to claim 6, wherein the top surface meets each side surface at an edge, and wherein the structural feature is offset from each edge of the top surface.

9. The image capturing module according to claim 6, wherein the structural feature surrounds the central region of the top surface.

10. The image capturing module according to claim 6, wherein the structural feature is a groove into the top surface.

11. The image capturing module according to claim 6, wherein the structural feature is a protrusion from the top surface.

12. The image capturing module according to claim 6, wherein the structural feature is a strip of material disposed on the top surface.

13. The image capturing module according to claim 1, wherein the surface modification includes an area of increased wettability to the resin, wherein the area of increased wettability is located at a periphery of the top surface.

14. The image capturing module according to claim 13, wherein the surface modification surrounds the central region of the top surface.

15. The image capturing module according to claim 1, wherein the surface modification includes an area having a first surface chemistry, wherein the surface modification surrounds the central region of the top surface, wherein the central region has a second surface chemistry, and wherein the first surface chemistry differs from the second surface chemistry.

16. The image capturing module according to claim 1, further comprising a wiring pattern, wherein the image sensing circuitry contacts the wiring pattern.

17. The image capturing module according to claim 16, wherein the image sensing circuitry contacts the wiring pattern via one or more solder joints.

18. An imaging unit (120), comprising a cartridge body (124) and the image capturing module (200) according to claim 1, wherein the image capturing module is seated in a recess of the cartridge body.

19. The imaging unit according to claim 18, wherein the imaging unit is configured to be attached to a distal end of an endoscope.

20. An endoscope, comprising the imaging unit according to claim 18.

21. An endoscope, comprising the image capturing module according to claim 1.

22. A method of manufacturing an image capturing module including an imager module with a lens and image sensing circuitry, the method comprising:
modifying a portion of a top surface of the lens to include a surface feature or a surface modification;
placing the imager module in a recess of a frame, the recess having a bottom surface and sidewalls; and
filling a gap between side surfaces of the lens and sidewalls of the frame with a resin,
wherein the resin contacts the surface feature or the surface modification, and
wherein the surface feature or the surface modification prevents the resin from flowing onto a central region of the top surface of the lens.

23. The method according to claim 22, wherein placing the imager module in the recess of the frame contacts the image sensing circuitry with a wiring pattern located within the recess.

24. The method according to claim 23, wherein the image sensing circuitry contacts the wiring pattern via one or more solder joints.

25. The method according to claim 22, wherein an area of the central region of the top surface of the lens is at least 80%, preferably at least 90%, more preferably at least 95%, of a total area of the top surface of the lens.

26. The method according to claim 22, wherein the top surface of the lens meets each side surface of the lens at an edge, wherein the surface feature includes a groove, and wherein modifying includes forming the groove at each edge of the top surface.

27. The method according to claim 26, wherein the groove surrounds the central region of the top surface of the lens.

28. The method according to claim 26, wherein the groove is formed by etching.

29. The method according to claim 26, wherein the surface feature includes a structural feature on the top surface of the lens.

30. The method according to claim 29, wherein the top surface of the lens meets each side surface of the lens at an edge, and wherein the structural feature is offset from at least one edge of the top surface.
